(19) United States
(12) Patent Application Publication
(10) Pub. No.: US 2025/0316368 A1
(43) Pub. Date: Oct. 9, 2025

(54) System and Method for Real-Time Pharmacy Inventory Synchronization and Integrated Communication to Improve Medication Access

(71) Applicant: Hayden Sklarow, Chantilly, VA (US)

(72) Inventor: Hayden Sklarow, Chantilly, VA (US)

(21) Appl. No.: 19/052,144

(22) Filed: Feb. 12, 2025

Related U.S. Application Data
(60) Provisional application No. 63/631,775, filed on Apr. 9, 2024.

(51) Int. Cl. G16H 40/20

(52) U.S. Cl. CPC G16H 40/20

(57) ABSTRACT

The present invention relates to a system and method for real-time synchronization of pharmacy inventory data and integrated communication between prescribers and pharmacists. The system employs a cloud-based architecture with middleware and API-based integrations to extract, standardize, and transmit inventory data from pharmacy management systems to hospital electronic health record systems. By maintaining continuous inventory updates and providing prescribers with real-time medication availability information, the system reduces prescription fulfillment delays, enhances operational efficiency, and improves patient access to necessary medications. The system supports both on-premise middleware and direct API integrations, ensuring compatibility with various pharmacy networks and healthcare systems.

### BACKGROUND OF THE INVENTION

### Field of Invention

The present invention relates generally to pharmacy inventory management and healthcare data synchronization. More specifically, the invention pertains to a system and method for real-time synchronization of pharmacy inventory data with healthcare provider systems, enabling seamless communication between prescribers and pharmacists. The invention is particularly applicable to enhancing medication accessibility, reducing prescription fulfillment delays, optimizing inventory management, and improving healthcare workflow efficiency. It further incorporates cloud-based infrastructure, real-time data synchronization, application programming interfaces (APIs), and integrated communication tools to facilitate improved medication availability, adherence, and regulatory compliance within the healthcare industry.

### BRIEF SUMMARY OF THE INVENTION

The present invention provides a system and method for real-time pharmacy inventory synchronization and integrated communication between healthcare providers and pharmacists. The system leverages cloud-based infrastructure, application programming interfaces (APIs), and middleware to enable continuous inventory updates, ensuring prescribers have accurate, real-time visibility into medication availability before issuing prescriptions. By integrating directly with pharmacy management systems (PMS) and electronic health records (EHRs), the invention eliminates inefficiencies associated with manual stock verification and reduces delays in medication access for patients.

In addition to inventory synchronization, the invention features an integrated communication platform that allows pharmacists to instantly notify prescribers of stock shortages, alternative medication options, or prior authorization requirements. This reduces reliance on time-consuming phone calls and manual interventions, streamlining prescription fulfillment and minimizing treatment delays. The system is designed to enhance medication adherence, reduce hospital readmissions due to prescription unavailability, and improve overall healthcare workflow efficiency through real-time data accuracy and seamless interoperability.

### BRIEF DESCRIPTION OF THE FIGURES

FIG. 1 illustrates the RealTimeRx pharmacy-hospital data flow, where pharmacy inventory data from a pharmacy management system (PMS) is transmitted through either installed software or API/data feeds into the RealTimeRx cloud, which then interfaces with the hospital electronic health record (EHR) system as the final step.

FIG. 2 depicts the pharmacy-to-cloud data flow, starting with pharmacy PMS or inventory software, which splits into three parallel processing paths: data extraction and transformation, encryption and security checks, and a local agent; the second split bypasses these steps and directly connects an API/data feed endpoint to the agent.

FIG. 3 shows the hospital EHR system requesting inventory data from the RealTimeRx cloud, which either loops back to the request source or moves to a validation and authorization process before leading to data retrieval and formatting, with the option to return the processed data to the RealTimeRx cloud.

### DETAILED DESCRIPTION

The present invention provides a system and method for real-time pharmacy inventory synchronization and integrated communication to address inefficiencies in medication access and prescription fulfillment. The system leverages cloud-based infrastructure, middleware, and application programming interfaces (APIs) to facilitate continuous and automated data exchange between pharmacy management systems (PMS) and electronic health records (EHRs). By implementing real-time or near real-time inventory updates, the system ensures that prescribers have accurate visibility into pharmacy stock levels before issuing prescriptions, reducing delays caused by unavailable medications. Unlike traditional methods that rely on periodic batch updates or manual stock verification, this invention eliminates outdated inventory discrepancies by maintaining a live data feed from participating pharmacies, thereby streamlining prescription processing and improving patient access to necessary treatments.

The system operates through a multi-layered integration framework that connects with PMS at the pharmacy level to extract, validate, and standardize inventory data. This data is transmitted securely to a cloud-based repository, where it undergoes automated processing to ensure accuracy, consistency, and compliance with regulatory standards. The inventory records are then made accessible to healthcare providers via EHR systems, enabling prescribers to query medication availability in real time. When a prescriber initiates a prescription order, the system retrieves the latest inventory data from the cloud repository, displaying stock availability across multiple pharmacy locations. The system can be configured to prioritize specific pharmacy networks, insurance-preferred providers, or patient-designated pharmacies, allowing prescribers to optimize medication fulfillment based on real-time stock levels and logistical considerations. This automated synchronization process significantly reduces instances where patients arrive at a pharmacy only to discover that their prescribed medication is unavailable, necessitating additional pharmacy visits or prescription modifications.

To further enhance prescription workflow efficiency, the system integrates a bidirectional communication module that enables pharmacists to notify prescribers of stock shortages, alternative medication options, and prescription-related issues such as prior authorization requirements, dosage clarifications, or therapeutic substitutions. This messaging functionality replaces conventional manual interventions such as phone calls or fax communications, which are time-consuming and prone to delays. The system supports structured message templates for common scenarios, enabling pharmacists to send instant notifications with actionable recommendations. For example, if a prescribed medication is out of stock, the pharmacist can propose a clinically equivalent alternative and request prescriber approval directly within the system. Additionally, the platform supports automated alerts for prescribers when inventory changes occur, reducing the likelihood of prescribing medications that are no longer available. By integrating communication directly into the prescription workflow, the system ensures that stock-related issues are resolved in real time, preventing unnecessary delays and improving medication adherence.

The cloud-based infrastructure is designed for scalability, supporting thousands of simultaneous pharmacy connections and high-frequency data transactions without compromising performance. To maintain data integrity, the system employs encryption protocols and role-based access controls, ensuring that sensitive inventory and prescription data is accessible only to authorized users. The platform also incorporates predictive analytics capabilities, which analyze historical inventory trends, seasonal demand fluctuations, and prescription patterns to forecast potential shortages. These insights enable pharmacies to proactively adjust stock levels before running out of high-demand medications, reducing the risk of supply chain disruptions and improving overall inventory management. Future enhancements to the system may include machine learning algorithms that refine predictive analytics over time, enabling even greater accuracy in anticipating pharmacy restocking needs.

In addition to supporting real-time inventory synchronization and communication, the invention aligns with healthcare regulatory requirements such as HIPAA and NCPDP standards, ensuring compliance with data privacy and interoperability guidelines. The system is designed to integrate seamlessly with existing healthcare IT ecosystems, including insurance formulary databases, prescription benefit managers (PBMs), and third-party medication tracking platforms. By maintaining compatibility with industry-standard protocols, the system facilitates nationwide scalability and adoption across diverse pharmacy networks, hospital systems, and outpatient care providers. Furthermore, the modular architecture allows for future expansion, such as integrating patient-facing applications that enable users to check medication availability at preferred pharmacies before filling their prescriptions.

Through its comprehensive approach to real-time inventory management and integrated prescriber-pharmacist communication, the present invention significantly improves the efficiency of medication dispensing workflows. By reducing prescription fulfillment delays, minimizing stock-related disruptions, and enhancing collaboration between healthcare providers, the system ultimately leads to better patient outcomes. Patients benefit from faster access to prescribed medications, prescribers experience fewer administrative burdens when managing medication availability, and pharmacies gain greater control over inventory accuracy and operational efficiency. The combination of real-time data synchronization, automated communication, and predictive inventory management represents a transformative advancement in pharmacy technology, addressing longstanding challenges in prescription fulfillment and medication accessibility.

### Scope of the System

The system connects two main types of endpoints:
Pharmacies—Inventory data is extracted from pharmacy management systems (PMS) or other inventory systems. Integration is achieved either through:
Custom middleware installed on-site
APIs provided by the pharmacy or third-party vendors
Hospital Systems (EHRs)—Hospital systems consume inventory data from the cloud to display real-time or near real-time availability of pharmaceutical products.

### Key Stakeholders

Pharmacy Owners and Operators—Provide inventory data for synchronization.
Hospitals and Healthcare Providers—Consume inventory data to improve operational efficiency.
System Administrators—Manage the deployment, maintenance, and troubleshooting of the system.
Software Developers and Architects—Design and implement the system's components.
Compliance Officers—Ensure the system adheres to regulatory standards (e.g., HIPAA, NCPDP).

### Objectives and Goals

Real-Time Inventory Synchronization—Ensure inventory data is updated in the cloud within a five-minute interval.
Seamless Integration—Support both custom on-premise middleware and API-based integrations
Scalability—Handle large volumes of data and simultaneous connections from multiple endpoints.
Security and Compliance—Protect sensitive data and ensure adherence to industry regulations.
Reliability—Minimize system downtime and ensure consistent data availability.

By achieving these objectives, the system aims to streamline inventory management, reduce operational inefficiencies, and improve decision-making across pharmacies and hospitals.

### System Overview

### High-Level Architecture Diagram

The system is composed of interconnected components that ensure seamless data flow between pharmacies, the cloud, and hospital systems. A high-level diagram (to be provided) outlines the following key components:
Pharmacy Integration Layer—Handles inventory data extraction from pharmacies via middleware or APIs.
Cloud Infrastructure—Processes, stores, and standardizes inventory data.
Hospital Integration Layer—Provides API endpoints for hospital systems to consume inventory data.

### Key Functional Components

Pharmacy Integration: Middleware for on-premise PMS systems, Cloud-based data synchronization for API-enabled systems, Transformation of raw data into standardized formats

Inventory Data Management: Centralized inventory database hosted in the cloud, Scheduled sync jobs (e.g., every 30 minutes) for near real-time data accuracy, Logging and monitoring for data integrity

Hospital Integration: RESTful API endpoints for inventory queries, Filter and query capabilities for hospital-specific needs, Caching mechanisms to improve response times and reliability

### System Interaction Overview

Pharmacy to Cloud: Data is extracted from PMS systems or received via APIs. The data is processed and synchronized with the cloud database

Cloud to Hospital: Hospitals query the cloud for inventory data. The system responds with relevant and up-to-date inventory information.

Error Handling and Notifications: Errors during synchronization or API calls are logged and flagged. Automated notifications are sent to system administrators for resolution.

This overview highlights how the system ensures reliable, scalable, and secure communication between endpoints.

### Pharmacy Integration

Local Integration with PMS Systems

For pharmacies that lack API capabilities, a custom middleware application will be installed on-site. This middleware connects directly to the pharmacy management system (PMS) or inventory database, extracting relevant inventory data and synchronizing it with the cloud.

Key features include:
Data Extraction—Using database queries, file parsing, or API hooks to retrieve inventory data.
Data Transformation—Converting extracted data into a standardized format compatible with the cloud database.
Sync Interval—Data is synchronized with the cloud every 30 minutes to ensure near real-time accuracy.
Error Logging—Local logs track synchronization issues, which are automatically reported to system administrators.
API—Based Integrations

For pharmacies with existing APIs or data feeds, the system will leverage these interfaces to synchronize inventory data.

Key considerations include:
API Compatibility—Supporting common standards like REST and SOAP.
Authentication—Using API keys, OAuth, or other mechanisms to ensure secure access.
Polling and Scheduling—Periodic polling of APIs (every 30 minutes or given provider limits) to retrieve the latest data.
Error Handling—Retries and fallback mechanisms in case of API failures.

### Data Transformation and Standardization

To ensure compatibility across systems, all incoming inventory data is transformed into a unified format.

This includes:
Normalization—Standardizing product identifiers (e.g., NDC codes, GPI codes, other classifications) and units of measurement.
Validation—Ensuring data integrity by checking for missing or inconsistent fields.
Metadata Enrichment—Adding supplemental information, such as timestamps and source identifiers, to each record.

### 3.4 Security and Compliance

Pharmacy integration adheres to strict security standards to protect sensitive data:
Encryption—Data is encrypted both in transit (using TLS) and at rest.
Access Control—Middleware and API access are restricted based on roles and permissions.
Compliance—The system aligns with HIPAA and other relevant regulations to ensure data privacy and security.

### Hospital Integration

### Cloud-Based Inventory Query

Hospital systems will interact with the platform via a RESTful API to request inventory data stored in the cloud.

Key features include:
Search and Filter Capabilities—Hospitals can query inventory based on parameters such as product name, NDC code, or availability.
Real-Time Data Access—Queries return the most up-to-date inventory data available.
Batch Requests—Support for bulk data retrieval to optimize performance for high-volume requests.

### API Specifications

The RESTful API will include endpoints for:
Inventory Lookup—Retrieve inventory details for specific products or categories.
Availability Check—Verify stock levels for high-priority items.
Custom Queries—Allow hospitals to specify complex filters (e.g., by expiration date or supplier).

Example endpoints:
GET/inventory
GET/inventory/{product_id}
GET/inventory/{inventory_type}/{product_id}
POST/inventory/query (for advanced filtering)

### Caching and Response Optimization

To ensure low-latency responses, the system will implement caching mechanisms:
Edge Caching—Frequently accessed queries are cached closer to the hospital systems.
TTL (Time-to-Live) Settings—Cached data is refreshed every 30 minutes to balance performance and data accuracy.
Priority Handling—Critical queries (e.g., urgent availability checks) bypass the cache to retrieve live data.

### DETAILED DESCRIPTION OF FIGURES

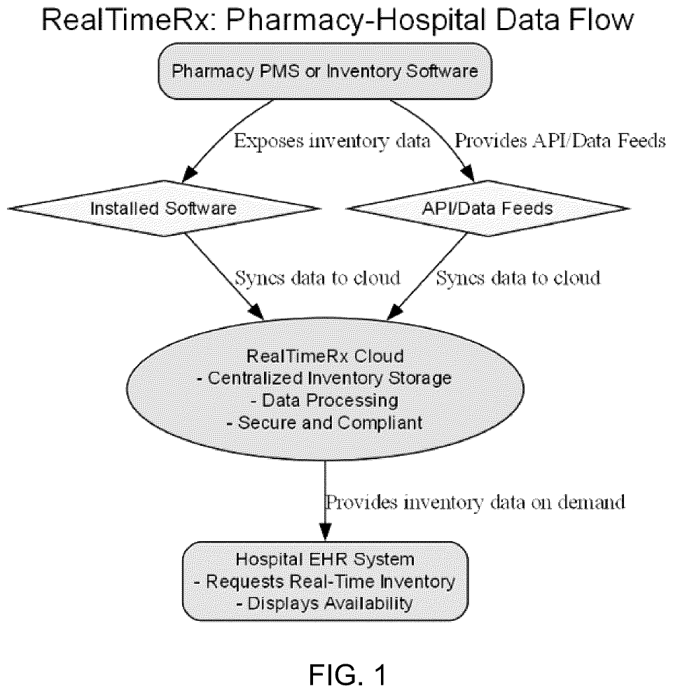

FIG. 1: RealTimeRx Pharmacy-Hospital Data Flow-illustrates the architecture and data flow of RealTimeRx as it synchronizes inventory data between pharmacies and hospitals. The process begins at the pharmacy PMS or inventory system, where real-time stock levels are maintained. From this point, data is transmitted using one of two pathways: installed software, which may include middleware for on-premise processing, or API/data feeds, which allow direct integration between the pharmacy system and RealTimeRx cloud services. Regardless of the transmission method, both pathways lead to the RealTimeRx cloud, where the incoming inventory data is processed, standardized, and stored. The hospital EHR system then accesses this synchronized inventory data, enabling prescribers to check real-time stock availability before issuing prescriptions. This final step ensures that healthcare providers have up-to-date medication availability insights, reducing prescription fulfillment delays and improving patient care.

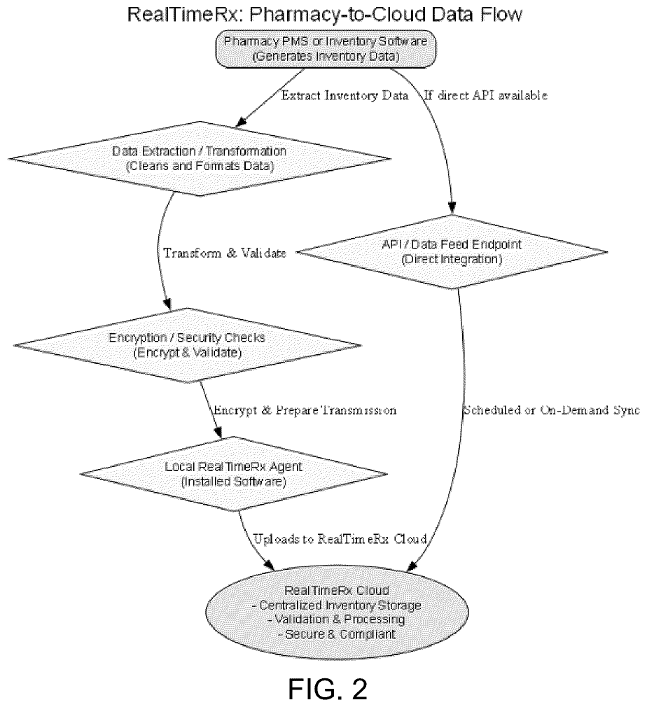

FIG. 2: Pharmacy-to-Cloud Data Flow-details the process of transmitting pharmacy inventory data to the RealTimeRx cloud. The process starts at the pharmacy PMS or inventory software, which maintains medication stock levels. From here, the data flow splits into two distinct processing paths. The first path includes data extraction and transformation, which processes raw pharmacy data into a standardized format, encryption and security checks, which ensure regulatory compliance and data integrity, and a local agent, which may handle on-site data processing before transmission. The second path bypasses these intermediary steps and sends the inventory data directly to an API/data feed endpoint, which then connects to the agent for secure transmission to the cloud. This parallel processing approach allows pharmacies to transmit data efficiently while ensuring security, compliance, and minimal latency.

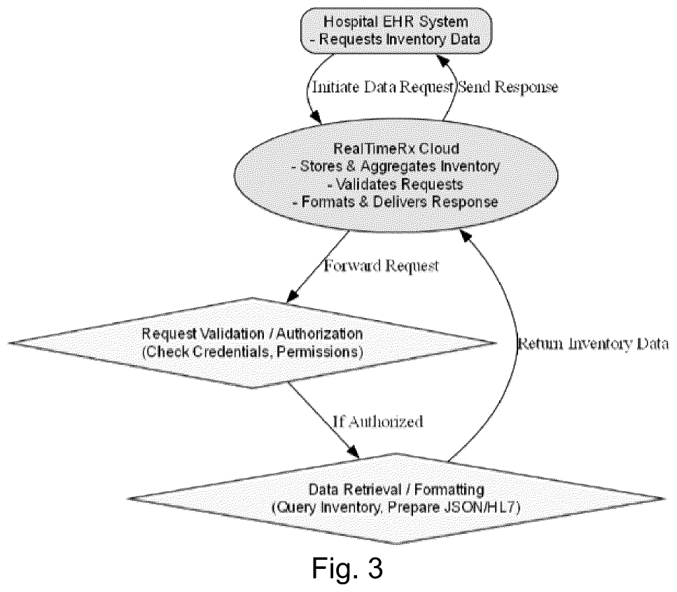

FIG. 3: Hospital EHR System Requesting Inventory Data-illustrates the process by which a hospital EHR system queries the RealTimeRx cloud to check medication availability. The request originates from the hospital EHR system, which submits an inventory query to the RealTimeRx cloud. The system then determines whether the request can be immediately fulfilled or requires further validation. If additional verification is needed, the request moves to a request validation and authorization step, where system rules, authentication measures, and access permissions are applied. Once validated, the request advances to data retrieval and formatting, where the inventory data is pulled from the RealTimeRx database, structured for compatibility with the hospital EHR system, and returned. The final step includes an option to either loop back to the RealTimeRx cloud for further data refinement or complete the transaction by delivering the formatted data to the hospital EHR. This architecture ensures secure and efficient inventory queries while maintaining data accuracy and regulatory compliance.

What is claimed is:

1. A system for real-time pharmacy inventory synchronization and integrated communication, comprising:
a. a cloud-based infrastructure configured to receive, process, and store inventory data from one or more pharmacy management systems (PMS);
b. a plurality of application programming interfaces (APIs) enabling bidirectional data exchange between said cloud-based infrastructure and electronic health records (EHRs) used by prescribers;
c. a synchronization engine configured to update medication availability in real time or near real time based on changes in pharmacy inventory levels;
d. a query module accessible by prescribers via EHRs to retrieve real-time inventory data from said cloud-based infrastructure; and
e. an integrated communication module configured to facilitate direct messaging between prescribers and pharmacists regarding stock shortages, alternative medications, prescription modifications, or prior authorization requirements.

2. The system of claim 1, wherein the synchronization engine retrieves inventory data from PMS at predefined intervals or upon detecting a stock change event to ensure continuous data accuracy.

3. The system of claim 1, wherein the query module allows prescribers to filter inventory data based on geographic location, pharmacy network affiliation, insurance formulary preferences, or patient-specific pharmacy selections.

4. The system of claim 1, wherein the integrated communication module supports automated alerts notifying prescribers of medication unavailability and suggesting clinically appropriate alternatives.

5. The system of claim 1, wherein the cloud-based infrastructure employs role-based access controls (RBAC) to restrict access to inventory data and communication features based on user credentials and permissions.

6. The system of claim 1, further comprising a predictive analytics engine that analyzes historical prescription trends, seasonal demand patterns, and pharmacy restocking behaviors to forecast potential medication shortages and recommend proactive inventory adjustments.

7. A method for real-time pharmacy inventory synchronization and prescriber-pharmacist communication, comprising:
a. receiving pharmacy inventory data from one or more PMS via APIs or middleware;
b. processing and standardizing the received inventory data within a cloud-based infrastructure;
c. updating real-time or near real-time inventory records to reflect stock availability;
d. enabling prescribers to query said inventory records via EHR systems to determine medication availability before issuing prescriptions;
e. providing prescribers with a list of pharmacies where the prescribed medication is in stock, based on real-time inventory data; and
f. facilitating bidirectional communication between prescribers and pharmacists to resolve stock shortages, suggest alternative medications, and address prescription-related issues.

8. The method of claim 7, wherein updating real-time inventory records further comprises receiving push notifications from pharmacy systems when stock changes occur.

9. The method of claim 7, wherein prescribers receive automatic alerts when a prescribed medication is unavailable, along with suggested alternative medications.

10. The method of claim 7, wherein bidirectional communication between prescribers and pharmacists includes structured message templates for common prescription modification scenarios, including dosage changes, therapeutic substitutions, and insurance prior authorization requirements.

11. The method of claim 7, further comprising integrating predictive analytics to forecast future medication demand based on historical prescription and inventory data trends.

12. The method of claim 7, further comprising securing inventory data using encryption protocols and role-based access controls to ensure compliance with healthcare data privacy regulations.

13. A computer-readable medium storing instructions that, when executed by a processor, cause a system to perform the method of claim 7.
